(19) United States
(12) Patent Application Publication
(10) Pub. No.: US 2009/0082582 A1
(43) Pub. Date: Mar. 26, 2009

(54) Process For Preparing [2-(2,3- Dihydrobenzofuran - Or Benzofuran-7-Yloxy)Ethyl]-(3 -Cyclopent-1-Ylbenzyl)Amine Derivatives and Synthesis Intermediate

(75) Inventors: Bernard Vacher, Castres (FR); Stephane Cuisiat, Castres (FR); Nicolas Roques, Brens (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Pierre Fabre Medicament, Boulogne (FR)

(21) Appl. No.: 12/225,048

(22) PCT Filed: Mar. 8, 2007
(86) PCT No.: PCT/FR2007/000479

§ 371 Date: Sep. 11, 2008

(30) Foreign Application Priority Data
Mar. 14, 2006 (FR) 0602194

(51) Int. Cl. C07D 317/20; C07D 307/79; C07C 51/00

(52) U.S. Cl. 549/453; 549/467; 562/492

(57) ABSTRACT

The invention relates to a process for the preparation of compounds of general formula (3)

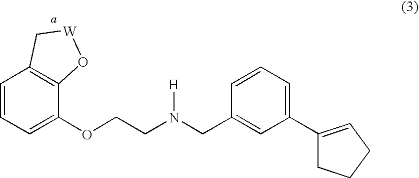

wherein:
(a) represents a single or double bond;
W represents a group CH, CH₂, CHCH₃, CCH₃, C(CH₃)₂, a group C(CH₂)₂ (i.e. a carbon atom carrying two methylene groups bonded to one another so as to form a spiro-cyclopropane moiety), with the proviso, however, that when (a) is a double bond then W represents exclusively a group CH or CCH₃ and when (a) is a single bond then W represents exclusively a group CH₂, CHCH₃, C(CH₃)₂ or C(CH₂)₂.

The present invention relates to a new process for the preparation of [2-(2,3-dihydro-benzofuran- or benzofuran-7-yloxy)-ethyl]-(3-cyclopenten-1-yl-benzyl)-amine derivatives of formula (3)

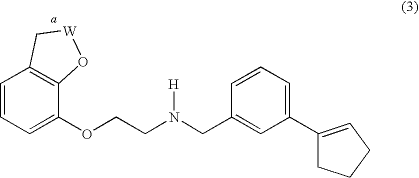

wherein: (a) represents a single or double bond; W represents a group CH, CH₂, CHCH₃, CCH₃, C(CH₃)₂, a group C(CH₂)₂ (i.e. a carbon atom carrying two methylene groups bonded to one another so as to form a spiro-cyclopropane moiety), with the proviso, however, that when (a) is a double bond then W represents exclusively a group CH or CCH₃ and when (a) is a single bond then W represents exclusively a group CH₂, CHCH₃, C(CH₃)₂ or C(CH₂)₂.

The compounds of formula (3), claimed in the International Application WO 2004/035561, are antagonists of dopaminergic receptors of the D₂ type and agonists of serotoninergic receptors of the 5-HT_{1A} sub-type. This double activity provides the compounds (3) with particular anti-psychotic properties both in animal models representative of the productive symptoms and in those representative of the deficit symptoms. The advantageous anti-psychotic properties of the compounds of formula (3) are moreover associated with a weak propensity to cause extra-pyramidal disturbances. By this token, the compounds of formula (3) are potentially useful in the treatment of acute and chronic psychotic states in humans. In view of their significant therapeutic potential and the considerable therapeutic need in this field, a process for the synthesis of the compounds (3) that is capable of industrial implementation is highly desirable.

The International Application WO 2004/035561 sets out a process for the preparation of the compounds (3). Said process involves a reductive amination reaction between the aldehyde of formula (1) and a primary amine of general formula (2), cf. Scheme A

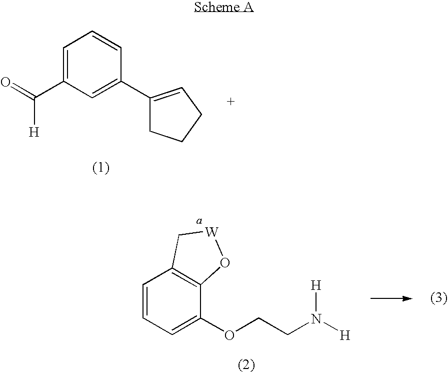

wherein (a) and W are as defined hereinbefore.

However, in the International Application WO 2004/035561, the aldehyde of formula (1) is prepared in three steps according to the sequence indicated in Scheme B.

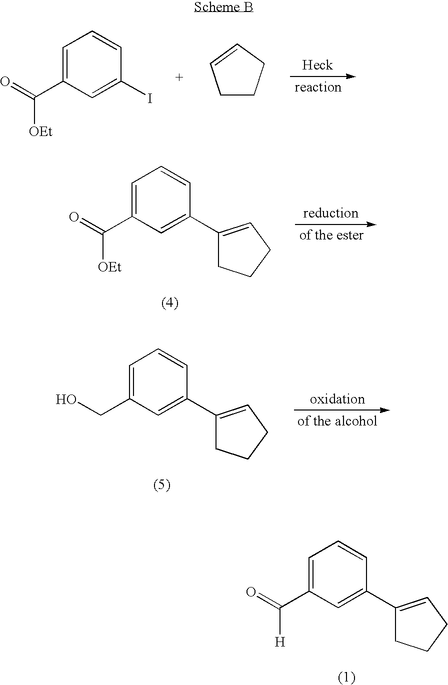

However, synthesis of the aldehyde (1) according to the route described in Scheme B has been found to be difficult to implement on an industrial scale, the first step in fact involving a Heck-type coupling reaction catalysed by a palladium complex. The use of transition metals poses the problem, however, both of their removal and of the extent to which the active ingredient (3) and also the effluents contain residual metals. In the particular case of the Heck reaction which results in the intermediate (4), the coupling is not entirely regioselective and a mixture of cyclopentene isomer compounds is obtained. Purification of the compound (4) is then carried out by chromatography over silica gel. This purification step becomes difficult to put into practice when the amounts of product to be purified become larger. Separation of the cyclopentene isomers at a later stage, for example from the compounds (5), (1) or (3), is not easy to carry out either. In addition, the moderate oxidation reaction of the alcohol (5) to the aldehyde (1) requires an excess of the oxidising agent (MnO₂) in order to obtain an acceptable level of conversion. At the end of the reaction, the aldehyde (1) formed is to a great extent adsorbed onto the surface of the precipitate, which has to be very carefully extracted, preferably in the hot state, in order for (1) to be recovered in an acceptable yield.

To sum up, the mode of preparation of the aldehyde (1) as described in WO 2004/035561 (Scheme B) is not satisfactory for implementation on a large scale and constitutes a limiting factor in obtaining the compounds of formula (3).

The present invention relates to a new process for the synthesis of the compounds (3). In accordance with the new process of the invention, the [2-(2,3-dihydro-benzofuran- or benzofuran-7-yloxy)-ethyl]-(3-cyclopenten-1-yl-benzyl)-amine derivatives of formula (3) are obtained by means of a reductive amination reaction as described in Scheme A starting from the intermediates (2) and (1), the aldehyde (1) used being obtained starting from the intermediate (6).

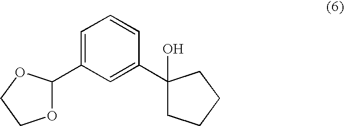

Preferably, for the synthesis of compounds of formula (3) there is used the aldehyde (1) obtained by deprotection and dehydration of 2-[3-(1-cyclopentane-1-hydroxy)phenyl]-1,3-dioxolane of formula (6).

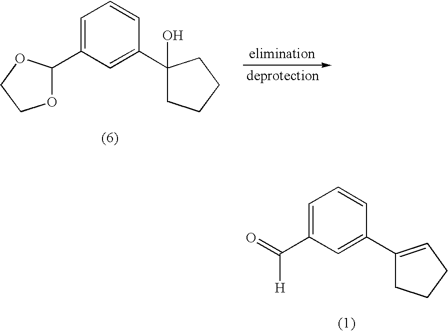

Even more preferably, the intermediate 6 is obtained by condensation of an organolithium compound, derived from 2-(3-bromophenyl)-1,3-dioxolane, with cyclopentanone.

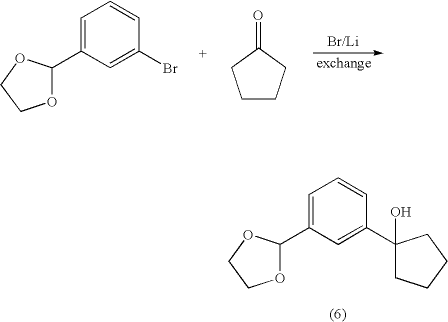

The present invention relates also to a new process for the synthesis of the aldehyde (1).

More specifically, the new process for the synthesis of the aldehyde (1) uses the tertiary alcohol of formula (6) as sole intermediate.

In accordance with the invention, the aldehyde of formula (1) is prepared in just two steps according to Scheme C in an overall yield which is very much higher than that obtained using the initial sequence (cf. Scheme B).

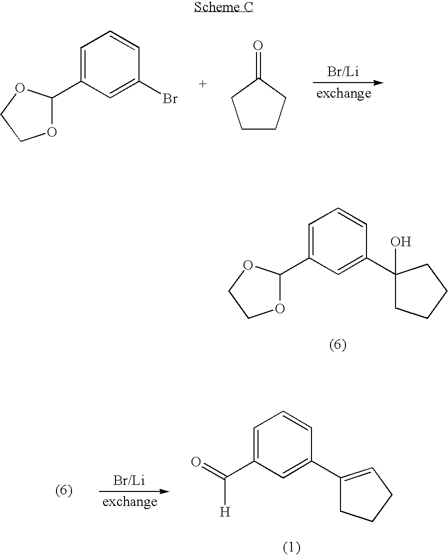

An essential aspect of the invention stems from the fact that the new process for the preparation of the aldehyde (1) no longer involves the oxidation step, the handling of which, it will be recalled, was especially problematic. An additional advantage of the invention lies in the fact that the synthesis of compound (I), and ultimately therefore of the active ingredient (3), is carried out without involving a catalyst based on transition metals.

The method of preparation of compound (I), in accordance with the invention, is described in detail hereinbelow.

The first step consists of condensing the aryllithium intermediate, derived from 2-(3-bromophenyl)-1,3-dioxolane [17789-14-9], with cyclopentanone [120-92-3], which is commercially available. The preparation of said aryllithium uses a bromine/lithium exchange reaction that is customary in organic chemistry (e.g. J. Med. Chem. 1998, 41, 358). In the case of interest to us, we have found that the presence of a Lewis acid minimised the formation of the reduction product (7) resulting from protonation of the aryllithium by the cyclopentanone. Lithium chloride has been found to be especially suitable for favouring the desired condensation reaction to the detriment of the reduction (cf. Scheme D). Under the conditions of the invention, the proportion of product (7) is in fact very low (<2%), which makes it possible to dispense with separation of the expected compound (6) from the by-product (7) by chromatography. It is of course advantageous to dispense with chromatographic separation, especially on a large scale.

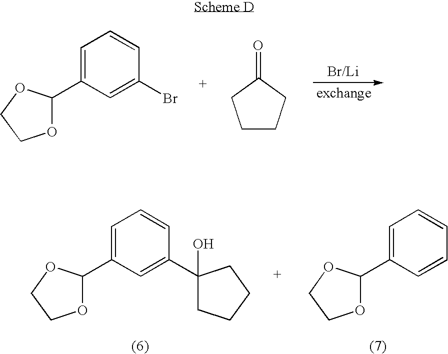

The second step combines two reactions: deprotection of the aldehyde function and dehydration of the tertiary alcohol. Separately, each of these reactions is well known to the person skilled in the art. There are also precedents, using substrates other than (6), involving these reactions concomitantly (e.g. J. Org. Chem. 1997, 62, 4183 and Org. Lett. 2000, 2, 1791). In the case of the intermediate (6), the operating conditions have been selected in order to carry out the double conversion as a “one-pot” procedure.

The process for the preparation of 3-(cyclopenten-1-ylphenyl)-carboxaldehyde of formula (1) as described hereinbefore is robust and practicable on the semi-industrial or industrial level. Accordingly, preparation of the compounds of formula (3) is found to be significantly improved with respect to the process described previously (WO 2004/035561).

Overall, the process for the synthesis of the compounds of formula (3) is, by virtue of the new method of obtaining the aldehyde (1), more advantageous on both the economic and the environmental level and thus is more favourable for industrial exploitation.

Another aspect of the invention relates to the intermediate of formula (6), i.e. 2-[3-(1-cyclopentane-1-hydroxy)phenyl]-1,3-dioxolane, a new compound identified, synthesised and used as intermediate in the synthesis of the aldehyde (1) and, ultimately, in the synthesis of the active compounds of formula (3).

The present invention relates also to a process for the synthesis of the intermediate of formula (6), i.e. 2-[3-(1-cyclopentane-1-hydroxy)phenyl]-1,3-dioxolane, by condensation of an aryllithium intermediate, derived from 2-(3-bromophenyl)-1,3-dioxolane, with cyclopentanone, preferably in the presence of a Lewis acid such as, for example, lithium chloride.

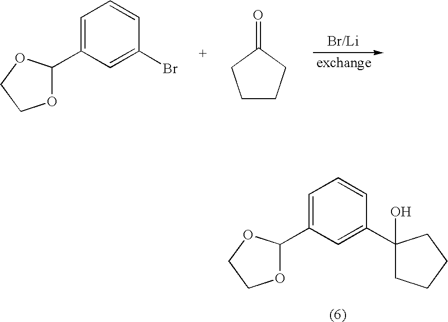

The Examples that follow illustrate the invention.

### EXAMPLE 1

### 2-[3-(1-cyclopentane-1-hydroxy)phenyl]-1,3-dioxolane (6)

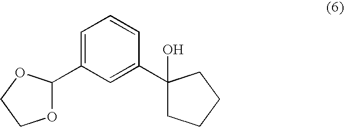

n-Butyllithium (2.5M in THF; 9.6 mL; 0.024 mol) is slowly added at −78° C. to a solution of 2-(3-bromophenyl)-1,3-dioxolane (5 gr, 0.022 mol) in dry THF (50 mL) and containing lithium chloride (1.85 gr; 0.043 mol). At the end of the addition, the reaction mixture is stirred for 1 hour 30 minutes at −78° C. and then cyclopentanone (2.9 mL; 0.033 mol) is added dropwise. The temperature is allowed to come back up to ambient temperature over two hours. The solution is then treated with water (10 mL), is diluted with ethyl acetate (100 mL) and is then washed with water and finally with a saturated aqueous solution of salt. The organic phase is dried over sodium sulfate, filtered and concentrated under reduced pressure. HPLC analysis of the organic phase reveals a (6)/(7) mixture of 98.5:1.5 (HPLC/Chrom Type: Fixed WL Chromatogram, 220 nm; column: Symmetry C8 5μ 250×4.6 mm Waters; eluant: acetonitrile/water, 1 mL/min; quantification of peaks by area).

The product (6) is isolated in the form of a colourless oil (3.7 gr, 72%). ¹H NMR (CDCl₃): □ 1.61 (s, 1H); 1.86 (m, 2H); 1.99 (m, 6H); 4.04 (m, 2H); 4.13 (m, 2H); 5.81 (s, 1H); 7.36 (m, 2H); 7.49 (m, 1H); 7.61 (s, 1H).

### EXAMPLE 2

### 3-(cyclopenten-1-ylphenyl)-carboxaldehyde (1)

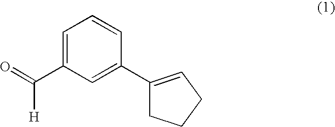

To a solution of 2-[3-(1-cyclopentane-1-hydroxy)phenyl]-1,3-dioxolane (6) prepared according to Example 1 (14.6 gr; 0.062 mol) in acetonitrile (250 mL) there is added, at 0° C., an aqueous solution of hydrochloric acid (4N; 63 mL; 0.25 mol). The reaction mixture is re-heated to ambient temperature and stirred for 16 hours. The mixture is then neutralised by pouring it into a saturated aqueous solution of sodium bicarbonate. The organic phase is concentrated under reduced pressure, the residue is taken up in ethyl acetate and the solution is washed with water and then with a saturated aqueous solution of salt. The organic phase is dried over sodium sulfate, filtered and concentrated under reduced pressure. The product is purified by flash chromatography over silica (cyclohexane/ethyl acetate, 95:5); 6.55 gr (61%) of product (1) are obtained in the form of a yellow oil.

¹H NMR (CDCl₃): □ 2.06 (m, 2H); 2.57 (m, 2H); 2.72 (m, 2H); 6.30 (s, 1H); 7.49 (t, J=7.6 Hz, 1H); 7.71 (m, 2H); 7.91 (s, 1H); 10.02 (s, 1H).

### EXAMPLE 3

### [2-(2,2-Dimethyl-2,3-dihydro-benzofuran-7-yloxy)-ethyl]-(3-cyclopenten-1-yl-benzyl)-amine (3a)

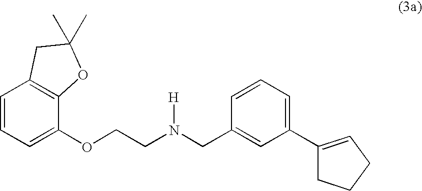

1.5 g of magnesium sulfate are added to a solution of 3-cyclopenten-1-yl-benzaldehyde (1a), prepared as in Example 2, (0.56 g, 3.26 mmol) and of [2-(2,2-dimethyl-2,3-dihydro-benzofuran-7-yloxy)]-ethylamine (2a), prepared according to WO 2004/035561, (0.68 g, 3.26 mmol) in 15 ml of 1,2-dichloroethane, and the mixture is heated at 60° C. for 17 hours. The mixture is cooled to ambient temperature, the solid is filtered off and the solvent is evaporated off under reduced pressure. The residue is diluted with 15 ml of methanol and then cooling to 0° C. is carried out. 0.35 g of potassium borohydride (6.52 mmol) is then added and the reaction mixture is stirred for three hours at 0° C. The mixture is then poured into iced water, extracted with ethyl acetate and washed with a saturated aqueous solution of sodium chloride. The combined organic phases are dried over magnesium sulfate and filtered, and the solvent is evaporated off under reduced pressure. The residue is purified by chromatography over silica gel (methylene chloride/methanol/ammonia: 98/1.5/0.5). The title product (0.61 g) is isolated in the form of a colourless oil.

¹H NMR (CDCl₃): δ 1.48 (s, 6H); 2.00 (m, 2H); 2.54 (m, 2H); 2.69 (m, 2H); 3.01 (s, 2H); 3.04 (t, J=5.6 Hz, 2H); 3.85 (s, 2H); 4.18 (t, J=5.6 Hz, 2H); 6.18 (s, 1H); 6.74 (m, 3H); 7.19 (d, J=7.4 Hz, 1H); 7.25 (t, J=8.7 Hz, 1H); 7.32 (d, J=7.6 Hz, 1H); 7.41 (s, 1H).

Fumarate of the compound of the title:

m.p.=146° C.

¹H NMR (DMSOd⁶): δ 1.39 (s, 6H); 1.96 (m, 2H); 2.51 (m, 2H); 2.65 (m, 2H); 2.96 (t, J=5.6 Hz, 2H); 2.99 (s, 2H); 3.91 (s, 2H); 4.11 (t, J=5.6 Hz, 2H); 6.27 (s, 1H); 6.56 (s, 2H); 6.71 (m, 1H); 6.79 (m, 2H); 7.31 (m, 2H); 7.37 (d, J=7.6 Hz, 1H); 7.50 (s, 1H);

IR (KBr) v: 3060, 2967, 1719, 1463 cm⁻¹;

Elemental analysis for C₂₄H₂₉NO₂.C₄H₄O₄

| | | | |
|---|---|---|---|
| theoretical %: | C 70.13 | H 6.94 | N 2.92 |
| found: | C 69.92 | H 6.93 | N 2.89. |

### EXAMPLE 4

### [2-(Benzofuran-7-yloxy)-ethyl]-(3-cyclopenten-1-yl-benzyl)-amine (3b)

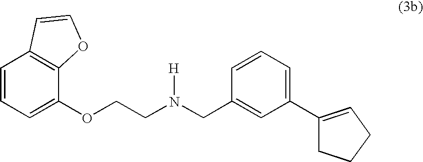

By proceeding as in Example 3 but using 2-(benzofuran-7-yloxy)ethyl-amine of formula (2b), prepared according to WO 2004/035561, instead of [2-(2,2-dimethyl-2,3-dihydro-benzofuran-7-yloxy)]-ethylamine of formula (2a), the title compound is obtained.

¹H NMR (CDCl₃): δ 2.04 (m, 2H); 2.53 (m, 2H); 2.70 (m, 2H); 3.12 (t, J=5.2 Hz, 2H); 3.90 (s, 2H); 4.33 (t, J=5.2 Hz, 2H); 6.19 (s, 1H); 6.76 (s, 1H); 6.83 (d, J=7.6 Hz, 1H); 7.13 (t, J=7.8 Hz, 1H); 7.19 (m, 2H); 7.29 (m, 2H); 7.33 (d, J=7.5 Hz, 1H); 7.44 (s, 1H); 7.61 (d, J=2.0 Hz, 1H).

Fumarate of the title product:

m.p.=126° C.

¹H NMR (DMSOd⁶): δ 1.95 (m, 2H); 2.49 (m, 2H); 2.64 (m, 2H); 3.04 (t, J=5.6 Hz, 2H); 3.91 (s, 2H); 4.29 (t, J=5.6 Hz, 2H); 6.26 (s, 1H); 6.57 (s, 2H); 6.93 (m, 2H); 7.15 (t, J=7.8 Hz, 1H); 7.26 (m, 3H); 7.36 (d, J=7.2 Hz, 1H); 7.49 (s, 1H); 7.95 (s, 1H);

IR (KBr) v: 3498, 2952, 2842, 1701, 1486 cm⁻¹;

Elemental analysis for C₂₂H₂₃NO₂.C₄H₄O₄

| | | | |
|---|---|---|---|
| theoretical %: | C 69.47 | H 6.05 | N 3.12 |
| found: | C 69.25 | H 6.08 | N 3.05. |

### EXAMPLE 5

### [2-(2,3-Dihydro-benzofuran-7-yloxy)-ethyl]-(3-cyclopenten-1-yl-benzyl)-amine (3c)

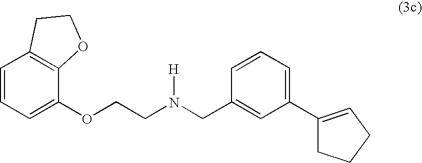

By proceeding as in Example 3 but using 2-(2,3-dihydro-benzofuran-7-yloxy)-ethyl-amine of formula (2c), prepared according to WO 2004/035561, instead of [2-(2,2-dimethyl-2,3-dihydro-benzofuran-7-yloxy)]-ethylamine of formula (2a), the title compound is obtained.

¹H NMR (DMSOd⁶): δ 1.95 (m, 2H); 2.40 (m, 2H); 2.65 (m, 2H); 2.81 (t, J=5.6 Hz, 2H); 3.13 (t, J=8.8 Hz, 2H); 3.74 (s, 2H); 4.03 (t, J=5.6 Hz, 2H); 4.46 (t, J=8.8 Hz, 2H); 6.25 (s, 1H); 6.79 (m, 3H); 7.27 (m, 3H); 7.42 (s, 1H).

Fumarate of the title product:

m.p.=118° C.

¹H NMR (DMSOd⁶): δ 1.92 (m, 2H); 2.49 (m, 2H); 2.65 (m, 2H); 2.93 (t, J=5.6 Hz, 2H); 3.16 (t, J=8.8 Hz, 2H); 3.88 (s, 2H); 4.11 (t, J=5.6 Hz, 2H); 4.50 (t, J=8.8 Hz, 2H); 6.27 (s, 1H); 6.56 (s, 2H); 6.81 (m, 3H); 7.24 (d, J=6.9 Hz, 1H); 7.30 (t, J=7.4 Hz); 7.36 (d, J=7.3 Hz, 1H); 7.48 (s, 1H);

IR (KBr) v: 3536, 3448, 2949, 2851, 1612, 1466 cm⁻¹;

Elemental analysis for C₂₂H₂₅NO₂.C₄H₄O₄

| | | | |
|---|---|---|---|
| theoretical %: | C 69.16 | H 6.47 | N 3.10 |
| found: | C 68.99 | H 6.55 | N 3.32. |

1-5. (canceled)

6. A process for the preparation of a [2-(2,3-dihydro-benzofuran- or benzofuran-7-yloxy)-ethyl]-(3-cyclopenten-1-yl-benzyl)-amine derivative selected from those of formula (3) 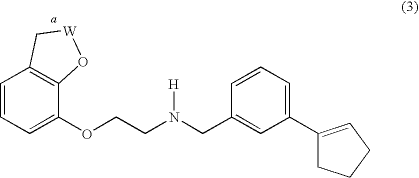
wherein:
(a) represents a single or double bond;
W represents CH, CH₂, CHCH₃, CCH₃, C(CH₃)₂, or C(CH₂)₂ (wherein the carbon atom and the two methylene groups bonded to one another so as to form a spiro-cyclopropane moiety),
provided that when (a) represents a double bond, W represents CH or CCH₃, and when (a) represents a single bond, W represents CH₂, CHCH₃, C(CH₃)₂ or C(CH₂)₂,
comprising the following steps:
a) preparation of 2-[3-(1-cyclopentane-1-hydroxy)-phenyl]-1,3-dioxolane of formula (6) by condensation of an aryllithium intermediate, derived from 2-(3-bromophenyl)-1,3-dioxolane, with cyclopentanone in accordance with the following reaction scheme: 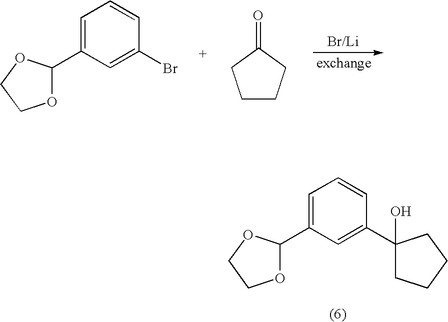
b) preparation of 3-(cyclopenten-1-ylphenyl)-carboxaldehyde of formula (1) by deprotection and dehydration of 2-[3-(1-cyclopentane-1-hydroxy)phenyl]-1,3-dioxolane of formula (6) as obtained in the preceding step 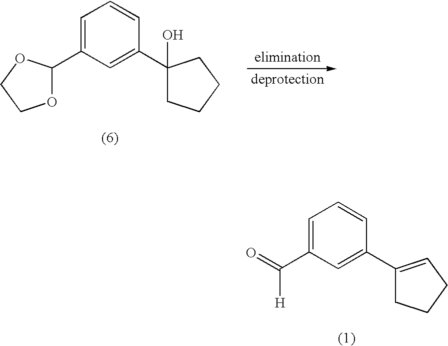
c) reductive amination reaction between a primary amine of general formula (2) 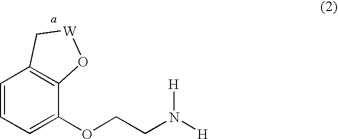
wherein:
(a) represents a single or double bond;
W represents CH, CH₂, CHCH₃, CCH₃, C(CH₃)₂, or C(CH₂)₂ (wherein the carbon atom and the two methylene groups bonded to one another so as to form a spiro-cyclopropane moiety),
provided that when (a) represents a double bond, W represents CH or CCH₃, and when (a) represents a single bond, W represents CH₂, CHCH₃, C(CH₃)₂ or C(CH₂)₂,
and 3-(cyclopenten-1-ylphenyl)-carboxaldehyde of formula (1) 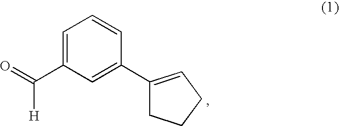
as obtained in the preceding step, to obtain the compound of formula (3).

7. 2-[3-(1-cyclopentane-1-hydroxy)phenyl]-1,3-dioxolane of formula (6)

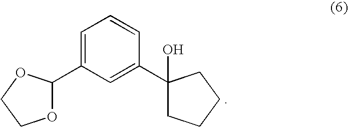

8. A process for the synthesis of 2-[3-(1-cyclopentane-1-hydroxy)phenyl]-1,3-dioxolane of formula (6) comprising condensation of an aryllithium intermediate, derived from 2-(3-bromophenyl)-1,3-dioxolane, with cyclopentanone in accordance with the following reaction scheme:

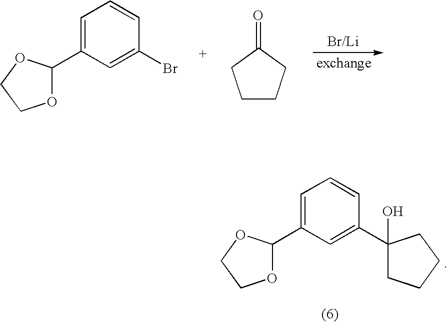

9. A process for the synthesis of 3-(cyclopenten-1-ylphenyl)-carboxaldehyde of formula (1) 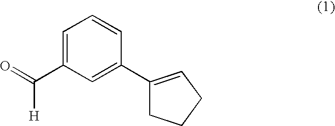
starting from 2-[3-(1-cyclopentane-1-hydroxy)phenyl]-1,3-dioxolane of formula (6).

10. The process according to claim 9, wherein 3-(cyclopenten-1-ylphenyl)-carboxaldehyde of formula (1) is obtained by deprotection and dehydration of 2-[3-(1-cyclopentane-1-hydroxy)phenyl]-1,3-dioxolane of formula (6) [chemical structure]
